(19) United States
(12) Patent Application Publication
(10) Pub. No.: US 2006/0275226 A1
(43) Pub. Date: Dec. 7, 2006

(54) Titanium dioxide and methylene-bis-benzotriazolyl-phenol mixture

(75) Inventors: Gerd Dahms, Duisburg (DE); Holger Seidel, Duisburg (DE); Olli-Pekka Antinluoma, Pori (FI); Riku Multisilta, Pori (FI)

Correspondence Address: BIRCH STEWART KOLASCH & BIRCH, PO BOX 747, FALLS CHURCH, VA, 22040-0747, US

(73) Assignee: KEMIRA PIGMENTS OY, Pori (FI)

(21) Appl. No.: 10/556,185

(22) PCT Filed: May 22, 2003
(86) PCT No.: PCT/EP03/05368

§ 371 Date: Jul. 25, 2006

(51) Int. Cl. A61K 8/49

(52) U.S. Cl. 424/59

(57) ABSTRACT

The mixture consists of c) from 5 to 60% by weight of titanium dioxide onto which organic and/or inorganic auxiliaries can be applied, as component A, d) from 40 to 95% by weight of an organic micropigment dispersion as component B, consisting of
b1) from 20 to 75% by weight of at least one methyleoebisbenzo-triazolylphenol in which the benzotriazolyl radicals and phenol radicals can each be independently substituted by one or more C₁₋₂₀ alkyl radicals, C₁₋₂₀ alkoxy radicals, C₁₋₂₀ hydroxyalkyl radicals, C₆₋₁₂ aryl radicals, halogen, hydroxyl groups or cyano groups, as organic micropigment B1, b2) from 2 to 15% by weight of at least one emulsifier B2, b3) from 0.5 to 5% by weight of organic auxiliaries B3, b4) from 22.5 to 77.5% by weight of water as component B4, the total amount of components B1 to B4 giving 100% by weight, with up to 20% by weight of the mixture being able to be replaced by other constituents.

The invention relates to mixtures of, preferably particulate, titanium dioxide and an organic micropigment dispersion based on a methylenebisbenzotriazolylphenol, the use of such mixtures for producing cosmetic OW, WO, PO or multiple emulsions, a process for production thereof and resultant WO emulsions. These are, in particular, sunscreen emulsions.

The harmful action of the ultraviolet portion of solar radiation on the human skin is generally known. Therefore, sunscreens which absorb the UV radiation in the UV-A and UV-B range have already been in production for a relatively long time.

Sunscreens currently generally comprise organic and, if appropriate, in addition inorganic, sunscreen filters. A UV filter having advantageous properties is 2,2′-methylenebis(6-(2H-benzotriazol-2-yl)-4-(1,1,3,3-tetramethylbutyl)phenol). This compound is marketed by Ciba Specialty Chemicals under the name Tinosorb® M as a 50% strength by weight aqueous dispersion. The dispersion appears as a white liquid which exhibits absorption maxima at 302 and 357 nm. The dispersion contains 50% by weight of methylenebisbenzotriazolyl-tetramethylbutylphenol, 7.5% by weight of decyl glucoside as emulsifier, 0.2% by weight of xanthan gums as thickener and 0.4% by weight of propylene glycol, and the remainder water. The organic pigment is present in this case in the form of microfine particles. The UV absorber acts here not only as micropigment but also as organic UV absorber. As a sunscreen filter it has a triple action due to UV absorption by a photostable organic molecule, due to light scattering and due to light reflection owing to the microfine structure. It is known that Tinosorb® M can be introduced into O/W emulsions, but not into W/O emulsions.

Even introduction into O/W emulsions causes difficulties and is subject to restrictions. DE-A-197 26 184 describes formulating Tinosorb® M in OW emulsions and OWO emulsions, with specific emulsifiers being used. It is stated that the preparations in addition can contain inorganic pigments, for example based on titanium dioxide. It is further stated that the inorganic pigments can be present in the form of micopigments. Amounts used are only stated to be generally from 0.1 to 30% by weight, preferably from 0.5 to 10% by weight, in particular from 1 to 6% by weight, based on the total weight of the preparations. No specific sequence in the formulation of the OW emulsions is mentioned. In particular, no formulation of a mixture of particulate titanium dioxide and Tinosorb® M is mentioned.

Similar OW emulsions which can contain, for example, 6% by weight of Tinosorb® M and 2% by weight of titanium dioxide are mentioned in DE-A-199 23 645. However, no specific combinations of Tinosorb® M and titanium dioxide are cited. The application is targeted in particular at the conjoint use of boron nitride to improve the feel of preparations on the skin.

DE-A-199 23 713 relates to similar systems which in addition have flavone derivatives and/or flavanone derivatives.

A frequently occurring problem in the use of Tinosorb® M in cosmetics formulations is achieving a sufficient degree of dispersion so that the active compound can develop optimum activity. For this reason, Tinosorb® M is used as a 50% strength by weight aqueous dispersion which has a high content of decyl glucoside as emulsifier or dispersant. The high amount of dispersant is necessary owing to its only partial efficacy.

As a consequence of this formulation of the organic micropigment, the production of cosmetic and other formulations is highly restricted, since the emulsifier available influences or impairs the production of the formulations. For example, WO emulsions cannot be produced, and OW emulsions can only be produced under certain circumstances and with limited starting materials.

The efficiency of the organic micropigment as a UV filter, however, depends on the type of formulation. If, owing to the original formulation of the organic micropigment having the high dispersant content, there are no or virtually no degrees of freedom for adjusting a finished formulation, optimization of efficiency is not possible.

The use of Tinosorb® M in WO formulations would be particularly advantageous, since WO formulations permit good and rapid film formation and have good water resistance. In addition, Tinosorb® M exhibits no decomposition under light irradiation and no skin penetration, as occurs in the case of liquid sunscreen filters.

It is an object of the present invention to provide a formulation for organic micropigment dispersions based on methylenebisbenzotriazolylphenol derivatives which avoids the disadvantages of known formulations and makes possible simple introduction into a multiplicity of formulations, so that optimization of formulations becomes possible.

The object is achieved according to the invention by a mixture consisting of
a) from 5 to 60% by weight of, preferably particulate, titanium dioxide onto which organic and/or inorganic auxiliaries can be applied, as component A,
b) from 40 to 95% by weight of an organic micropigment dispersion as component B, consisting of
   b1) from 20 to 75% by weight of at least one methylenebisbenzo-triazolylphenol in which the benzotriazolyl radicals and phenol radicals can each be independently substituted by one or more C₁₋₂₀ alkyl radicals, C₁₋₂₀ alkoxy radicals, C₁₋₂₀ hydroxyalkyl radicals, C₆₋₁₂ aryl radicals, halogen, hydroxyl groups or cyano groups, as organic micropigment B1,
   b2) from 2 to 15% by weight of at least one emulsifier B2,
   b3) from 0.5 to 5% by weight of organic auxiliaries B3,
   b4) from 22.5 to 77.5% by weight of water as component B4,
   the total amount of components B1 to B4 giving 100% by weight, with up to 20% by weight of the mixture being able to be replaced by other constituents.

It has been found according to the invention that by adding, preferably particulate, titanium dioxide, in particular microfine titanium dioxide having a particle size in the range from 5 to 500 nm, the ability to formulate the organic micropigment dispersions is considerably improved. In the use of the inventive mixture, OW, WO, PO or multiple emulsions can be produced and optimized in a broad range of compositions, without dispersion problems with regard to the organic micro-pigment occurring.

Adding the, preferably particulate, titanium dioxide thus improves the ability to formulate the said organic micropigment very considerably, so that performance-optimized compositions are accessible. The optimization relates here, in particular, to the light protection action in sunscreens which are applied to the human skin.

The inventive mixture consists of the components A and B, in which case up to 20% by weight, preferably up to 10% by weight, in particular up to 5% by weight, of the mixture can be replaced by other constituents. According to a preferred embodiment, the mixture consists of only the components A and B.

Component A is present in the inventive mixtures in an amount from 5 to 60% by weight, preferably from 10 to 40% by weight, in particular from 15 to 30% by weight. Component A is, preferably particulate, titanium dioxide to which organic and/or inorganic aids can be applied. The, preferably particulate, titanium dioxide preferably has here a mean particle size in the range from 5 to 500 nm, particularly preferably from 10 to 80 nm. In particular it is microfine titanium dioxide. The titanium dioxide can be used in the customary crystal forms in untreated or surface-treated form. A surface treatment with organic and/or inorganic aids is possible. Organic aids which can be used are, in particular, organic polymers. Inorganic aids can be metal oxides and/or nonmetal oxides.

Particularly preferably, only non-surface-treated titanium dioxide is present in component A.

Component B is present in the inventive mixtures in an amount of from 40 to 95% by weight, preferably from 60 to 90% by weight, in particular from 70 to 85% by weight. Component B is an organic micropigment dispersion which consists of the components B1 to B4, the total amount of which gives 100% by weight. Component B1 is present in an amount of from 20 to 75% by weight, preferably from 40 to 60% by weight. It is at least one methylenebisbenzotriazolylphenol, in which the benzotriazole radicals and phenol radicals can each independently be substituted by one or more C₁₋₂₀ alkyl radicals, C₁₋₂₀ alkoxy radicals, C₁₋₂₀ hydroxyalkyl radicals, C₆₋₁₂ aryl radicals, halogen, hydroxal groups or cyano groups. Preferably, only the phenol radicals are monosubstituted or disubstituted, the substituent preferably being in p position to the hydroxyl group. Particularly preferably, the phenol radicals are substituted by a C₄₋₁₂ alkyl radical in the p position. In particular, component B1 is 2,2′-methylenebis(6-(2H-benzotriazol-2-yl)-4-(1,1,3,3-tetramethylbutyl)phenol), as is present in the formulation Tinosorb® M from Ciba Specialty Chemicals.

As component B2, from 2 to 15% by weight, preferably from 5 to 10% by weight, of at least one emulsifier are present. Suitable emulsifiers are the emulsifiers specified hereinafter. In particular, alkyl glucosides or alkyl polyglucosides are used. Especially, C₆₋₂₀ alkyl glucosides, in particular decyl glucosides, are used.

As component B3, from 0.5 to 5% by weight, preferably from 0.5 to 1% by weight, organic aids are present. These are preferably thickeners and organic solvents. The thickeners used can be the thickeners listed below, in particular xanthan gums. One example of a suitable organic solvent is propylene glycol. For example, 0.2% by weight of xanthan gums and 0.4% by weight of propylene glycol are present.

As a remaining component B4, water is present in an amount such that the total amount of the components gives 100% by weight. The amount of component B4 is generally from 22.5 to 77.5% by weight, preferably from 30 to 50% by weight.

The preferred component B used is the dispersion marketed as Tinosorb® M by Ciba Specialty Chemicals.

According to the invention, the, preferably particulate, titanium dioxide of the component A is used as formulation aid for the organic micropigment dispersions B in the production of cosmetic OW, WO, PO or multiple emulsions.

In particular, the microfine titanium dioxide further disperses itself in the formulations, so that a microfine pigment distribution of the organic and inorganic pigments is present. The dispersion equilibrium of the organic micropigments is not influenced and is not impaired here.

In the case of simultaneous use of the titanium dioxide with the organic micropigment in sunscreens, a synergistic effect results, which goes far beyond an additive sunscreen action for the components.

A synergistic effect of this type has been found to date only for the combination of Tinosorb® M with octyl methoxycinnamate (OMC). According to the invention, a synergistic sunscreen effect has now been found for the combination of the solid UV filter with titanium dioxide.

As a result, the inventive mixtures can be used particularly advantageously in the production and formulation of cosmetic emulsions or sunscreen emulsions. The invention also relates to a process for producing OW, WO, PO or multiple emulsions which contain organic micropigment dispersions, a mixture described as above being introduced into the OW, WO, PO or multiple emulsions and then homogenized.

The invention also relates to a WO emulsion which comprises an oil phase, a water phase and, based on the total emulsion, from 1 to 50% by weight, preferably from 3 to 15% by weight, of the above-described mixture.

In the production of the OW, WO, PO or multiple emulsions, the inventive mixture can be used directly, or the components A and B of the inventive mixture can be introduced separately into the emulsions. It is possible in this case to introduce the components A and B or their mixture into one or more of the phases used for producing the emulsions and then to mix the individual phases. On the other hand, it is also possible according to the invention first to produce the finished emulsion without the inventive mixture and then to introduce the mixture or the components A and B into the emulsion.

Further suitable constituents of the emulsion are described below. Reference may also be made to the corresponding disclosures in DE-A-197 26 184, DE-A-109 23 645, DE-A-199 23 713 and DE-A-109 33 461.

The emulsions can be present as high-viscosity systems such as gels. It is possible to produce emulsions, lotions, gels, liposomes for all customary sectors, such as pharmaceutical, cosmetic, dermatological, food engineering or laundry detergent sectors. Other fields of application are also accessible according to the invention. The emulsions can be macroemulsions, microemulsions or nanoemulsions. Nanoemulsions have emulsion droplets having a mean diameter in the range from 5 to 1 000 nm, preferably from 15 to 300 nm.

The inventive mixtures can also be used to produce aqueous active-compound carrier nanodispersions which contain at least one pharmaceutical or cosmetic active compound. Active compound carrier particles used in this case are lipid-based particles. These include lipids and lipid-like structures. Examples of suitable lipids are mono-, di- and triglycerides of saturated straight-chain fatty acids having from 12 to 30 carbon atoms, such as lauric acid, myristic acid, palmitic acid, stearic acid, arachidic acid, behenic acid, lignoceric acid, cerotic acid, melesic acid, and esters thereof with other polyhydric alcohols such as ethylene glycol, propylene glycol, mannitol, sorbitol, saturated fatty alcohols having from 12 to 22 carbon atoms, such as lauryl alcohol, myristyl alcohol, cetyl alcohol, stearyl alcohol, arachidyl alcohol, behenyl alcohol, saturated waxy alcohols having from 24 to 30 carbon atoms such as lignoceryl alcohol, ceryl alcohol, ceratyl alcohol, myristyl alcohol. Preference is given to mono-, di-, triglycerides, fatty alcohols, esters or ethers thereof, waxes, lipopeptides or mixtures thereof. In particular, synthetic mono-, di- and triglycerides are used as individual substances, or in the form of a mixture, for example in the form of a hard fat. Glycerol trifatty acid esters, are, for example, glycerol trilaurate, glycerol trimyristate, glycerol palmitate, glycerol tristearate or glycerol tribehenate. Suitable waxes are, for example, cetyl palmitate and Cera alba (bleached wax, DAB [German Pharmacopoeia] 9). As lipids, polysaccharides, polyalkyl acrylates, polyalkyl cyanoacrylates, polyalkylvinylpyrrolidones, acrylic polymers, polylactic acids or polylactides can also be used.

The amount of the active compound carrier particles, based on the total aqueous active compound carrier dispersion is preferably from 0.1 to 30% by weight, particularly preferably from 1 to 10% by weight. In addition to the lipids, dispersion stabilizers can also be used. They can be used, for example, in amounts of from 0.01 to 10% by weight, preferably from 0.05 to 5% by weight. Examples of suitable substances are surfactants, in particular ethoxylated sorbitan fatty acid esters, block polymers and block copolymers (for example poloxamers and poloxamines), polyglycerol ethers and polyglycerol esters, lecithins of various origin (for example egg or soya lecithin), chemically modified lecithins (for example hydrogenated lecithin) and also phospholipids and sphingolipids, mixtures of lecithins with phospholipids, sterols (for example cholesterol and cholesterol derivatives and also stigmasterol), esters and ethers of sugars or sugar alcohols with fatty acids or fatty alcohols (for example sucrose monostearate), sterically stabilizing substances such as poloxamers and poloxamines (polyoxyethylene-polyoxypropylene block polymers), ethoxylated sorbitan fatty acid esters, ethoxylated mono- and diglycerides, ethoxylated lipids and lipoids, ethoxylated fatty alcohols or fatty acids and charge stabilizers or charge carriers, for example dicetyl phosphate, phosphatidylglycerol and saturated and unsaturated fatty acids, sodium cholate, sodium glycocholate, sodium taurocholate or mixtures thereof, amino acids or peptizing agents such as sodium citrate (see J. S. Lucks, B. W. Muller, R. H. Müller, Int. J. Pharmaceutics 63, pages 183 to 189 (1990)), viscosity-increasing substances such as cellulose ethers and cellulose esters (for example methylcellulose, hydroxyethylcellulose, hydroxypropylcellulose, sodium carboxymethylcellulose), polyvinyl derivatives such as polyvinyl alcohol, polyvinylpyrrolidone, polyvinyl acetate, alginates, polyacrylates (for example Carbopol), xanthans and pectins.

The aqueous phase used can be water, aqueous solutions or mixtures of water with water-miscible liquids, such as glycerol or polyethylene glycol. Other additional components for the aqueous phase are, for example, mannose, glucose, fructose, xylose, trehalose, mannitol, sorbitol, xylitol or other polyols such as polyethylene glycol, and electrolytes such as sodium chloride. These additional components can be used in an amount of from 0.5 to 60, for example from 1 to 30,% by weight, based on the aqueous phase A.

If wanted, further viscosity-increasing substances or charge carriers can be used, as are described in EP-B-0 605 497.

Emulsifiers which form lamellar structures which can be used are natural or synthetic products. It is also possible to use surfactant mixtures. Examples of suitable emulsifiers are the physiological bile salts such as sodium cholate, sodium dehydrocholate, sodium deoxycholate, sodium glycocholate, sodium taurocholate.

Animal and plant phospholipids such as lecithins together with their hydrogenated forms and also polypeptides such as gelatin with its modified forms can also be used.

Synthetic surface-active substances which are suitable are the salts of sulphosuccinic esters, polyoxyethylene acid betan esters, acid betan esters and sorbitan ethers, polyoxyethylene fatty alcohol ethers, polyoxyethylene stearic acid esters and corresponding mixture condensates of polyoxyethylene methpolyoxypropylene ethers, ethoxylated saturated glycerides, partial fatty acid glycerides and polyglycides. Examples of suitable surfactants are Biobase® EP and Ceralution® H.

Examples of suitable emulsifiers are, in addition, glycerol esters, polyglycerol esters, sorbitan esters, sorbitol esters, fatty alcohols, propylene glycol esters, alkyl glucosite esters, sugar esters, lecithin, silicone copolymers, lanolin and mixtures or derivatives thereof. Glycerol esters, polyglycerol esters, alkoxylates and fatty alcohols and also isoalcohols can be derived, for example, from castor fatty acid, 12-hydroxystearic acid, isostearic acid, oleic acid, linoleic acid, linolenic acid, stearic acid, myristic acid, lauric acid and capric acid. In addition to the said esters, succinates, amides or ethanolamides of the fatty acids can also be present. Fatty acid alkoxylates which can be used are, in particular, the ethoxylates, propoxylates or mixed ethoxylates/propoxylates.

To prepare the inventive cosmetic emulsions, generally emulsifiers are used. Examples of suitable emulsifiers are glycerol esters, polyglycerol esters, sorbitan esters, sorbitol esters, fatty alcohols, propylene glycol esters, alkyl glucoside esters, sugar esters, lecithin, silicone copolymers, lanolin and mixtures and derivatives thereof. Glycerol esters, polyglycerol esters, alkoxylates and fatty alcohols and isoalcohols can be derived, for example, from rhizinus fatty acid, 12-hydroxystearic acid, isostearic acid, oleic acid, linoleic acid, linolenic acid, stearic acid, myristic acid, mauric acid and capric acid. In addition to the said esters, succinates, amides or ethanolamides of the fatty acids can also be present. Fatty acid alkoxylates which can be used are, in particular, the ethoxylates, propoxylates or mixed ethoxylates/propoxylates. In addition, emulsifiers can be used which form lamellar structures. Examples of such emulsifiers are the physiological bile salts such as sodium cheolate, sodium dehydrocheolate, sodium deoxycheolate, sodium glycochealate, sodium taurochealate. Animal and plant phospholipids such as lecithins together with their hydrogenated forms, and polypeptides such as gelatin together with its modified forms can also be used.

Suitable synthetic surface-active substances are the salts of sulphosuccinic esters, polyoxyethylene acid bethan esters, acid bethan esters and sorbitan ethers, polyoxyethylene fatty alcohol ethers, polyoxyethylenestearic acid esters and corresponding mixture condensates of polyoxyethylene-methpolyoxypropylene ethers, ethoxylated saturated glycerides, partial fatty acid glycerides and polyglycides. Examples of suitable surfactants are Biobase® EP and Ceralution® H.

Lipids and emulsifiers are preferably used in a weight ratio of from 50:1 to 2:1, preferably from 15:1 to 30:1.

The additional pharmaceutical, cosmetic and/or food-engineering active compounds are preferably used in an amount of from 0.1 to 80% by weight, particularly preferably from 1 to 10% by weight.

The further active compounds are, according to one embodiment of the invention, additional sunscreen filters. These can be present as organic sunscreen filters in liquid or solid form at room temperature (25° C.). Suitable sunscreen filters (UV filters) are, for example, compounds based on benzophenone, diphenyl cyanoacrylate or p-aminobenzoic acid. Specific examples are (INCI or CTFA names) Benzophenone-3, Benzophenone-4, Benzophenone-2, Benzophenone-6, Benzophenone-9, Benzophenone-1, Benzophenone-11, Etocrylene, Octocrylene, PEG-25 PABA, Phenylbenzimidazole Sulfonic Acid, Ethylhexyl Methoxycinnamate, Ethylhexyl Dimethyl PABA, 4-Methylbenzylidene Camphor, Butyl Methoxydibenzoylmethane, Ethylhexyl Salicylate, Homosalate and also 2-hydroxy-4-methoxybenzophenone-5-sulphonic acid and 2,4,6-trianilino-p-(carbo-2′-ethylhexyl-1′-oxy)-1,3,5-triazine.

Other organic sunscreen filters are Octyltriazone, Avobenzone, Octylmethoxycinnamate, Octyl Salicylate, Benzotriazole and Triazine.

Further possible constituents of the emulsions are additional hydrophilically coated micropigments, electrolytes, glycerol, polyethylene glycol, propylene glycol, barium sulphate, alcohols, waxes, metal soaps, magnesium stearate, Vaseline or other constituents. For example, in addition, fragrances, fragrance oils or fragrance aromas can be added. Suitable cosmetic active compounds are, for example, polyphenols and compounds derived therefrom. Suitable vitamins are retinol, tocopherol, ascorbic acid, riboflavin and pyridoxin.

All known and suitable water-in-oil emulsions or oil-in-water emulsions can be produced using the inventive mixture. For this, they can be used after the described emulsifiers and other constituents. In addition, the production of polyol-in-oil emulsions is possible. In this case any suitable polyols can be used.

In the emulsions, the proportions of the two main phases can be varied within broad ranges. For example, from 5 to 95% by weight, preferably from 10 to 90% by weight, in particular from 20 to 80% by weight, of the respective phases are present, in which case the total amount gives 100% by weight.

The P/O emulsion described can also be emulsified in water or a water-in-oil emulsion. This results in a polyol-in-oil-in-water emulsion (P/O/W emulsion), which contains at least one described emulsion and in addition at least one aqueous phase. Such multiple emulsions can correspond in structure to the emulsions described in DE-A-43 41 113.

On introducing the P/O emulsion into water or aqueous systems, the weight ratio of the individual phases can be varied within broad ranges. Preferably, in the P/O/W emulsion finally obtained, the percentage by weight of the P/O emulsion is from 0.01 to 80% by weight, particularly preferably from 0.1 to 70% by weight, in particular from 1 to 30% by weight, based on the entire P/O/W emulsion.

When the P/O emulsion is introduced into an O/W emulsion, the content of P/O emulsion is preferably from 0.01 to 60% by weight, particularly preferably from 0.1 to 40% by weight, in particular from 1 to 30% by weight, based on the P/O/W emulsion finally obtained. In the O/W emulsion which is used for this purpose, the oil content is preferably from 1 to 80% by weight, particularly preferably from 1 to 30% by weight, based on the O/W emulsion used. Instead of a P/O emulsion, a W/O emulsion can also be introduced, which leads to a W/O/W emulsion. The individual phases of the emulsions can further have known constituents usual for the individual phases. For example, the individual phases can further comprise pharmaceutical or cosmetic active compounds which are soluble in these phases. The aqueous phase can comprise, for example, additional organic soluble sunscreen filters, additional hydrophilically coated micropigment, electrolytes, alcohols, etc. Individual phases or all of the phases can also comprise additional solids which are preferably selected from pigments or micropigments, microspheres, silica gel and similar substances. The oil phase can comprise, for example, additionally organically modified clay minerals, hydrophobically coated (micro)pigments, organic oil-soluble sunscreen filters, oil-soluble active cosmetic compounds, waxes, metal soaps such as magnesium stearate, Vaseline or mixtures thereof (Micro)pigments which may be mentioned are titanium dioxide, zinc oxide and barium sulphate, and also wollastonite, kaolin, talc, Al₂O₃, bismuth oxychloride, micronized polyethylene, mica, ultramarine, eosin dyes, azo dyes.

The water phase can, furthermore, comprise glycerol, polyethylene glycol, propylene glycol, ethylene glycol and similar compounds and derivatives thereof.

The use of customary aids and additives in the emulsions is known to those skilled in the art.

The aqueous phase which can be used is water, aqueous solutions or mixtures of water with water-miscible liquids, such as glycerol or polyethylene glycol. In addition, electrolytes such as sodium chloride may be present in the aqueous phase. If wanted, in addition, viscosity-increasing substances or charge carriers can further be used, as are described in EP-B-0605 497.

The invention will now be described in more detail by the examples below.

### EXAMPLES

In the examples hereinafter, the pigment dispersion TinosorbV M from Ciba Specialty Chemicals is used, as discussed in the above description together with the constituents.

### Example 1

The organic pigment dispersion Tinosorb® M was mixed with differing finely divided titanium dioxide powders. The particle size distribution was then determined by laser light scattering. The compositions and the resultant mean particle sizes are listed in the table below.

WV Titan X111 is an aluminium oxide-modified titanium dioxide having a particle size of about 14 nm. The specific surface area is about 100 m²/g.

TABLE 1
Particle size analyses of various aqueous pigment dispersions

| | Mean particle | Proportion of | Specific particle |
|---|---|---|---|
| Pigment dispersed | diameter | particles <1 μm | surface area |
| in water | median [μm] | [%] | [cm2/cm3] |
| UV Titan M212 | 0.37 | 95.2 | 125632 |
| UV Titan X111 | 0.36 | 77.5 | 16432 |
| Tinosorb M | 0.28 | 100 | 215085 |
| Tinosorb m + UV | 0.37 | 95.2 | 125632 |
| Titan M212 | | | |
| Tinosorb M + UV | 0.29 | 99 | 132053 |
| Titan X111 | | | |

The results of the table show that when Tinosorb® M is mixed with the titanium dioxide particles, a mean particle size distribution was established which was in the middle of the means of both particle size distributions for Tinosorb® M and titanium dioxide pigment alone.

The width of the distributions was not changed by combining Tinosorb® M and titanium dioxide particles.

### Example 2 (comparison)

OW sunscreens, formulated with TiO₂ or Tinosorb ® M

| | | | 2a | 2b |
|---|---|---|---|---|
| | | | [% by | [% by |
| Trade name | Manufacturer | CTFA/INCI | weight] | weight] |
| Phase A | | | | |
| Tegocare 450 | Goldschmidt | Polyglyceryl-3-methyl glucose distear | 5.00 | 5.00 |
| Miglyol 812 N | Sasol | Caprylic/capric triglyceride | 21.00 | 21.00 |
| Phase B | | | | |
| Demin. water | — | Aqua | 67.70 | 55.70 |
| Ceralution F | Sasol | Sodium lauroyl lactylate, sodium dico | 1.00 | 1.00 |
| Keltrol | NutraSweet | Xanthan gum | 0.30 | 0.30 |
| Phase C | | | | |
| UV-Titan X111 | Kemira | Titanium dioxide, alumina | 4.00 | 0.00 |
| Tinosorb M | Ciba | Methylene Bis-Benzotriazolyl Tetram | 0.00 | 16.00 |
| Phase D | | | | |
| Phenonip | Nipa | Phenoxyathanol, methylparaben, eth | 1.00 | 1.00 |
| Total | | | 100.00 | 100.00 |

For production, phases A and B were heated separately to 70° C. Phase A was added to phase B and the mixture was homogenized. It was then cooled to 30° C. Phase C was added to the mixed phase AB, and this mixture was homogenized. Finally phase D was added to phase ABC and the final mixture was stirred slowly.

Both compositions were subjected to a stability test. The results are summarized in the following table.

| Stability test | 2 a | 2 b |
|---|---|---|
| RT | | |
| 3 days | 1 | 1 |
| 1 week | 1 | 1 |
| 2 weeks | 1 | 1 |
| 40° C. | | |
| 1 day/3 days | 1 | 1 |
| 5 days | 1 | 1 |
| 1 week | 1 | 4/O 5% |
| 50° C. | | |
| 1 day/3 days | 1 | 1 |
| 5 days | 1 | 1 |
| 1 week | 1 | 5 |
| −18° C./40° C. | | |
| 1× | 1 | 1 |
| 2× | 1 | 1 |
| 3× | 1 | 3/O |
| 4× | 1 | 3/O |
| 5× | | 3/O |

Observation:
1 = OK
2 = inhomogeneous
3/W = visible water separation
3/O = visible oil separation
4/W = measurable water separation
4/O = measurable oil separation
5 = total separation

### Example 3

OW sunscreens formulated with a mixture of TiO₂ and Tinosorb® M

| | | | 3 |
|---|---|---|---|
| Trade name | Manufacturer | CTFA/INCI | [% by weight] |
| Phase A | | | |
| Ceralution H | Sasol | Sodium lauroyl lactylate, sodium | 4.00 |
| | | dicocoylethylenediamine PEG-15 | |
| | | sulphate | |
| Miglyol 812 N | Huls | Caprylic/capric triglyceride | 21.00 |
| Phase B | | | |
| Demin. water | | | 40.90 |
| Ceralution F | Sasol | Sodium lauroyl lactylate, sodium | 1.00 |
| | | dicocoylethylenediamine PEG-15 sulphate | |
| Keltrol | Kelco | Xanthan gum | 0.30 |
| Phase C | | | |
| Tinosorb M | Ciba | Methylenebisbenzotriazolyl- | 16.00 |
| | | tetramethylbutylphenol | |
| UV-Titan X111 | Kemira | Titanium dioxide, alumina | 4.00 |
| Phase D | | | |
| Carbopol ETD | B F Goodrich | Carbomer | 10.00 |
| 2050 (3%) | | | |
| Phase E | | | |
| 10% NaOH | | | 1.80 |
| Phase F | | | |
| Phenonip | Nipa | Phenoxyethanol, methylparaben, | 1.00 |
| | | ethylparaben, butylparaben, | |
| | | propylparaben, isobutylparaben | |
| Total: | | | 100.00 |

The sunscreens were produced by separately heating phases A and B to 70° C., subsequently adding phase A to phase B, and homogenizing. The mixture was then cooled to 30° C., and phase C (X111 predispersed in Tinosorb® M) was added to phase AB. This mixture was then homogenized. Phase D was then introduced with stirring, and the mixture was neutralized with phase E. Phase F was then introduced with a spatula, with stirring.

The composition had an in-vivo Sun Protection Factor of 18.7.

The formulations of Examples 2 and 3, in the context of storage tests, showed that incorporating Tinosorb® M into OW formulations leads to instability. This instability is due to the action of the Tinosorb® M dispersion aid (APG). APG, as a hydrophilic surfactant, is incorporated into the liquid-crystal gel network of the OW emulsion and destabilizes it. Therefore, the formulation of Example 2b, which only contained Tinosorbg M, also proved incapable of being stored.

In comparison, incorporating the inorganic pigment, and incorporating the mixture of Tinosorb® M and inorganic pigment, did not lead to destabilization; see Examples 2a and 3.

The stability of the formula of Example 3 was tested at 50° C. for 3 months and for 5 tan-freeze cycles. The stability of the emulsion was retained for this period and number of tan-freeze cycles.

### Example 4

WO-sunscreens, formulated with a mixture of TiO₂ and Tinosorb® M

| Trade name | Manufacturer | CTFA/INCI | [% by weight] |
|---|---|---|---|
| Phase A | | | |
| Abil WE | Goldschmidt | Polyglyceryl-4-isostearate, | 5.00 |
| | | cetyldimethicone copolyl, hexyl | |
| | | laurate | |
| Asmisol 110 | Lucas Meyer | Lecithin, oleamide DEA, soybean | 1.25 |
| | GmbH | oil | |
| Finsolv TN | | | 12.00 |
| Arlamol HD | Uniqema | Heptamethylnonane | 5.00 |
| Cetiol SN | Cognis | Cetearyl isononanoate | 12.00 |
| Vitamin E Acetate | Roche | Tocopheryl acetate | 1.00 |
| Phase B | | | |
| Demin. water | | | 41.35 |
| Propylenglycol | Overlack | Propylene glycol | 2.00 |
| Natriumchlorid | Merck | Sodium chloride | |
| Phase C | | | |
| UV-Titan X111 | Kemira | | 4.00 |
| TINOSORB M | Ciba | Methylenebisbenzotriazolyl- | 16.00 |
| | | tetramethylbutylphenol | |
| Total: | | | 100.00 |

To produce the sunscreens, phase B was added to phase A, and the mixture was homogenized. Phase C was then added to phase AB, and this mixture was homogenized.

The sunscreen had an in-vivo Sun Protection Factor of 13.4.

### Example 5

WO-sunscreens, formulated with TiO₂ /Tinosorb ® M

| | 5a | 5b | 5c |
|---|---|---|---|
| | % by weight | (comp.) | (comp.) |
| Tradename | HS | BB | BB |
| Phase A | | | |
| Abil WE 09 | 5.00 | 5.00 | 5.00 |
| Amisol 110 | 1.25 | 1.25 | 1.25 |
| Finsolv TN | 12.00 | 12.00 | 12.00 |
| Arlamol HD | 5.00 | 5.00 | 5.00 |
| Cetiol SN | 12.00 | 12.00 | 12.00 |
| Vitamin E Acetat | 1.00 | 1.00 | 1.00 |
| Phase B | | | |
| Demin. water | 41.35 | 57.35 | 45.35 |
| Propylenglycol | 2.00 | 2.00 | 2.00 |
| Tinosorb M | 16.00 | 0.00 | 16.00 |
| UV-Titan X111 | 4.00 | 4.00 | 0.00 |
| Natriumchlorid | 0.40 | 0.40 | 0.40 |
| Total: | 100.00 | 100.00 | 100.00 |

The sunscreens were produced by dispersing the pigment of phase B in Tinosorb® M and diluting with water. Phase B was then slowly added to phase A, and the mixture was homogenized for one minute.

For Example 5a, an in-vitro Sun Protection Factor of 21.9 was found.

The stability test in accordance with Example 2 gave the following results:

| | 5a | 5b | 5c |
|---|---|---|---|
| RT | | | |
| 1 day | 1 | 1 | 1 |
| 3 days | 1 | 1 | 1 |
| 1 week | 1 | 1 | 1 |
| 2 weeks | 1 | 1 | 1 |
| 3 weeks | 1 | | |
| 1 month | 1 | | |
| 2 months | 1 | | |
| 3 months | 1 | | |
| 40° C. | | | |
| 1 day | 1 | 1 | 4/W 10% |
| 3 days | 1 | 1 | 4/W 10% |
| 1 week | 1 | 1 | — |
| 2 weeks | 1 | 1 | — |
| 3 weeks | 4/O 10% | | — |
| 50° C. | | | |
| 1 day | 1 | 1 | 4/W 30% |
| 3 days | 1 | 1 | — |
| 1 week | 1 | 1 | — |
| 2 weeks | 3/O | 1 | — |
| 3 weeks | 3/O | | — |
| 4 weeks | 3/O | | — |
| 2 months | 4/O 20% | | — |
| 3 months | 4/O 20% | | — |
| −18° C./40° C. | | | |
| 1× | 1 | 1 | 1 |
| 2× | 1 | 1 | 4/W 5% |
| 3× | 1 | 1 | 4/W 5% |
| 4× | 3/O | 1 | 4/W 5% |

1-10. (canceled)

11. Mixture consisting of
c) from 10 to 40% by weight of particulate of titanium dioxide having a particle size in the range from 5 to 500 nm onto which organic and/or inorganic auxiliaries can be applied, as component A,
d) from 60 to 90% by weight of an organic micropigment dispersion as component B, consisting of
b1) from 20 to 75% by weight of at least one methylenebisbenzotriazolylphenol in which the benzotriazolyl radicals and phenol radicals can each be independently substituted by one or more C₁₋₂₀ alkyl radicals, C₁₋₂₀ alkoxy radicals, C₁₋₂₀ hydroxyalkyl radicals, C₆₋₁₂ aryl radicals, halogen, hydroxyl groups or cyano groups, as organic micropigment B1,
b2) from 2 to 15% by weight of at least one emulsifier B2,
b3) from 0.5 to 5% by weight of organic auxiliaries B3,
b4) from 22.5 to 77.5% by weight of water as component B4,
the total amount of components B I to B4 giving 100% by weight,
with up to 20% by weight of the mixture being able to be replaced by other constituents, obtainable by adding component A to component B.

12. Mixture according to claim 11, characterized in that the organic micropigment is a methylenebisbenzotriazolyl (C₄₋₁₂-alkylphenol).

13. Mixture according to claim 11, characterized in that component B consists of from 40 to 60% by weight of component B1, from 5 to 10% by weight of component B2, from 0.5 to 1% by weight of thickeners and organic solvents as component B3 and from 30 to 50% by weight of component B4, the total amount of components B I to B4 giving 100% by weight.

14. Process for stabilizing cosmetic OW- or WO-emulsions containing organic micropigment dispersions B as defined in claim 11, involving the step of adding particulate titanium dioxide having a particle size in the range from 5 to 500 nm to which organic and/or anorganic aids can be applied, to the OW- or WO-emulsion as formulation aid for the organic micropigment dispersion B.

15. Process for producing OW, WO, PO or multiple emulsions which comprise methylenebisbenzotriazolylphenols in which the benzotriazole radicals and phenol radicals comprise can each independently be substituted by one or more C₁₋₂₀ alkyl radicals, C₁₋₂₀ alkoxy radicals, C₁₋₂₀ hydroxyalkyl radicals, C₆₋₁₂ aryl radicals, halogen, hydroxyl groups or cyano groups, characterized in that a mixture according to claim 11 is introduced into the OW, WO, PO or multiple emulsions and this mixture is then homogenized.

16. Process according to claim 15, characterized in that the emulsion is a cosmetic emulsion or sunscreen emulsion.

17. Mixture according to claim 12, characterized in that component B consists of from 40 to 60% by weight of component B1, from 5 to 10% by weight of component B2, from 0.5 to 1% by weight of thickeners and organic solvents as component B3 and from 30 to 50% by weight of component B4, the total amount of components B1 to B4 giving 100% by weight.
